(19) United States
(12) Patent Application Publication
(10) Pub. No.: US 2021/0043305 A1
(43) Pub. Date: Feb. 11, 2021

(54) MEDICAL IMAGE DIAGNOSIS SYSTEM, MEDICAL IMAGE PROCESSING METHOD, AND STORAGE MEDIUM

(71) Applicant: CANON KABUSHIKI KAISHA, Tokyo (JP)

(72) Inventor: Ryo Tanaka, Kawasaki-shi (JP)

(21) Appl. No.: 16/943,629

(22) Filed: Jul. 30, 2020

(30) Foreign Application Priority Data
Aug. 5, 2019 (JP) 2019-143768

(51) Int. Cl. G16H 30/40; G06T 11/60; G16H 50/20

(52) U.S. Cl. CPC G16H 30/40; G16H 50/20; G06T 11/60

(57) ABSTRACT

A medical image diagnosis system includes a generation unit that generates a medical image associated object relating to a medical image, a first associating unit that associates the medical image associated object with the medical image, a grouping unit that assigns a plurality of annotations to be added to the medical image to two or more groups, and a second associating unit that associates each of the two or more groups with the medical image associated object. The grouping unit assigns one of the plurality of annotations to a plurality of groups among the two or more groups.

### BACKGROUND

### Field

The present disclosure relates to a medical image diagnosis system, a medical image processing method, and a storage medium.

### Description of the Related Art

In recent years, radiological technologists roles have started to include assistance in image interpretation. For example, when there is no radiologist or specialist available, the radiological technologist who regularly reviews medical images is expected to point out any abnormalities. The radiological technologist can use graphics, characters, and symbols called “annotations” in order to notify a doctor of items that the radiological technologist noticed during imaging and in regard to the medical image.

The annotations are stored together with the medical image as overlay data or a Grayscale Softcopy Presentation State (GSPS) that conforms to the Digital Imaging and Communications in Medicine (DICOM) standard. When the doctor observes the medical image, the medical image and the overlay data or the GSPS are read by a medical image display apparatus, and all the annotations are displayed on the medical image in a superimposed manner. Even annotations for other purposes are displayed together in a superimposed manner. A method of selectively displaying annotations suitable for a purpose in such a case is discussed in Japanese Patent Application Laid-Open No. 2013-132514, which discloses a technology in which annotations are grouped by a medical image display apparatus to be stored for each group.

Some annotations are common to a plurality of groups. Such annotations common to a plurality of groups are required to be assigned to each group, which can lead to an increase in time and labor.

### SUMMARY

Aspects of the present disclosure has been made in view of the above, and are directed to provide a technology for efficiently grouping annotations based on purposes.

According to at least one embodiment, a medical image diagnosis system includes a generation unit configured to generate a medical image associated object relating to a medical image, a first associating unit configured to associate the medical image associated object with the medical image, a grouping unit configured to assign a plurality of annotations to be added to the medical image to two or more groups, and a second associating unit configured to associate each of the two or more groups with the medical image associated object, wherein the grouping unit is configured to assign one of the plurality of annotations to a plurality of groups among the two or more groups.

Further features will become apparent from the following description of exemplary embodiments with reference to the attached drawings.

### BRIEF DESCRIPTION OF THE DRAWINGS

FIG. 1 illustrates an example of a configuration of an imaging system.

FIG. 2A illustrates a display example of a new inspection input screen.

FIG. 2B illustrates a display example of the new inspection input screen.

FIG. 2C illustrates a display example of the new inspection input screen.

FIG. 3 illustrates a display example of an imaging screen.

FIG. 4 illustrates a display example of a key object creation screen.

FIG. 5 illustrates a display example of the imaging screen.

FIG. 6 is a flow chart illustrating a GSPS creation process.

FIG. 7 illustrates an example of an annotation group.

### DESCRIPTION OF THE EMBODIMENTS

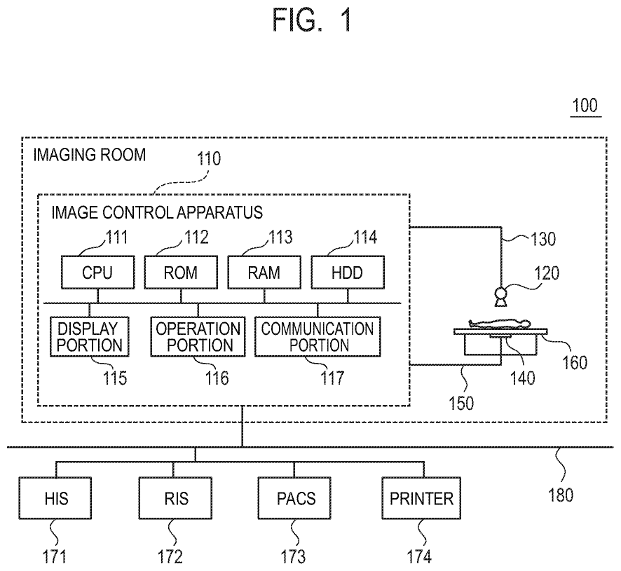

FIG. 1 illustrates an example of a configuration of an imaging system 100 according to an exemplary embodiment. The imaging system 100 is an example of a medical image diagnosis system. The imaging system 100 performs imaging of a radiation image as an example of a medical image. The imaging system 100 includes an imaging control apparatus 110, a hospital information system (HIS) 171, and a radiology information system (RIS) 172. The imaging system 100 also includes a picture archiving and communication system (PACS) 173 and a printer 174.

The imaging control apparatus 110 manages radiation imaging. The HIS 171 manages progress of radiation imaging. The HIS 171 can include a server that manages information for e.g., hospital accounting. When it is determined that radiation imaging for a patient is required, an operator inputs an inspection instruction through the HIS 171. The inspection instruction is transmitted to a radiology department as the requested destination. This request information is referred to as “inspection order”. The inspection order includes the name of a department being a request source, an inspection item, and personal data on a patient.

When receiving the inspection order via the MS 172, the radiology department adds, for example, imaging conditions to the inspection order, and transfers the inspection order to the imaging control apparatus 110. The imaging control apparatus 110 executes radiation imaging based on the received inspection order. Inspection information is added to an obtained image, and the obtained image is transferred to the PACS 173 and/or printed out by the printer 174. In addition, execution information on an inspection by the imaging control apparatus 110 is transferred to the HIS 171. The execution information transferred to the HIS 171 is used for progress management of the inspection as well as for a hospital accounting process after the inspection.

The imaging control apparatus 110, and the HIS 171, RIS 172, PACS 173, and printer 174 are connected to each other via a network 180 that is, for example, a local area network (LAN) and a wide area network (WAN), and each include one or a plurality of computers. The one or plurality of computers include a main controller, for example, a CPU, and storage components such as, for example, a read only memory (ROM) and a random access memory (RAM). The computer can also include a communication component such as, for example, a network card, and an input/output component such as, for example, a keyboard, a display, or a touch panel. These components are connected to each other via, for example, a bus, and are controlled by the main controller reading and executing a program stored in the storage component.

The imaging control apparatus 110 will now be described. The imaging control apparatus 110 includes a CPU 111, a ROM 112, a RAM 113, an HDD 114, a display portion 115, an operation portion 116, and a communication portion 117. The CPU 111 reads a control program stored in the ROM 112 to execute various processes. The RAM 113 is used as a temporary storage area, such as a main memory or a work area of the CPU 111. The HDD 114 stores, for example, various kinds of data and various programs.

The display portion 115 displays various kinds of information. The display portion 115 is, for example, a liquid crystal display. The operation portion 116 includes a button or a mouse, and receives various operations performed by a user. The display portion 115 and the operation portion 116 can be implemented as a touch panel in which both the display portion 115 and the operation portion 116 can are integrated.

The communication portion 117 performs a communication process to/from an external apparatus via a wired or wireless connection. The communication portion 117 is connected to, for example, a radiation generation unit 120 via a cable 130. The communication portion 117 is connected to, for example, a radiation detector 140 via a cable 150. The communication portion 117 is also connected to the HIS 171 and some of the other above-described components via the network 180.

The functions and processes of the imaging control apparatus 110 described below are implemented by the CPU 111 reading programs stored in the ROM 112 or the HDD 114 to execute the programs. In another embodiment, the CPU 111 can read a program stored in a recording medium, for example, an SD card, in place of a program stored in the ROM 112 or another storage component.

The radiation generation unit 120 is implemented by, for example, a radiation tube bulb, and irradiates an object, e.g., a patient's specific body part with radiation. The radiation generation unit 120 is controlled by the CPU 111. The radiation detector 140 functions as a detector configured to detect the radiation that has passed through the object to acquire a radiation image based on the object (hereinafter referred to simply as “radiation image”). That is, the radiation generation unit 120 and the radiation detector 140 cooperate with each other to implement a radiation imaging portion. The radiation detector 140 is installed on an imaging table 160 in an upright position or in a lying position.

The CPU 111 instructs starting of radiation imaging corresponding to at least one piece of order information received from the RIS 172. Each piece of order information includes, for example, information on a subject to be examined and one or a plurality of the subject's body parts to be examined to be imaged. The CPU 111 is assumed to receive a start instruction based on a user operation performed via the operation portion 116. In another embodiment, the CPU 111 can select a piece of order information to instruct to start imaging. When imaging is performed, an image is displayed on the display portion 115. The operator can perform image editing, which includes image processes, clipping, addition of annotations, and geometric conversion, on the displayed image via the operation portion 116.

The hardware configuration of the imaging control apparatus 110 is not limited to the configuration described in the present embodiment. In another exemplary embodiment, at least a part of the functions and the processes of the imaging control apparatus 110 can be implemented by causing a plurality of CPUs, RAMs, ROMs, and storages to cooperate with each other. In still another exemplary embodiment, at least a part of the functions and processes of the imaging control apparatus 110 can be implemented via use of a hardware circuit. Specifically, a CPU configured to control radiation generation and a CPU configured to control imaging can be provided to the imaging control apparatus 110.

The configuration of an imaging control system is not limited to the above-described configuration. For example, in FIG. 1, various apparatus are connected to the imaging control apparatus 110 via the network 180, but the imaging control apparatus 110 is not required to be connected to such apparatuses. A diagnostic image can be output to a portable storage medium, for example, a DVD, and can be input to various apparatuses via the portable storage medium. The network 180 can be a wired network or can be partially a wireless signal transmission line.

A processing procedure performed when the imaging system 100 is used to take a radiation image along an inspection flow will now be described. An operator first inputs patient information and inspection information to the imaging control apparatus 110 based on an inspection request form or an inspection request received from the RIS 172. The patient information includes a patient name and a patient ID. The inspection information includes imaging information for defining details of imaging to be performed on the patient.

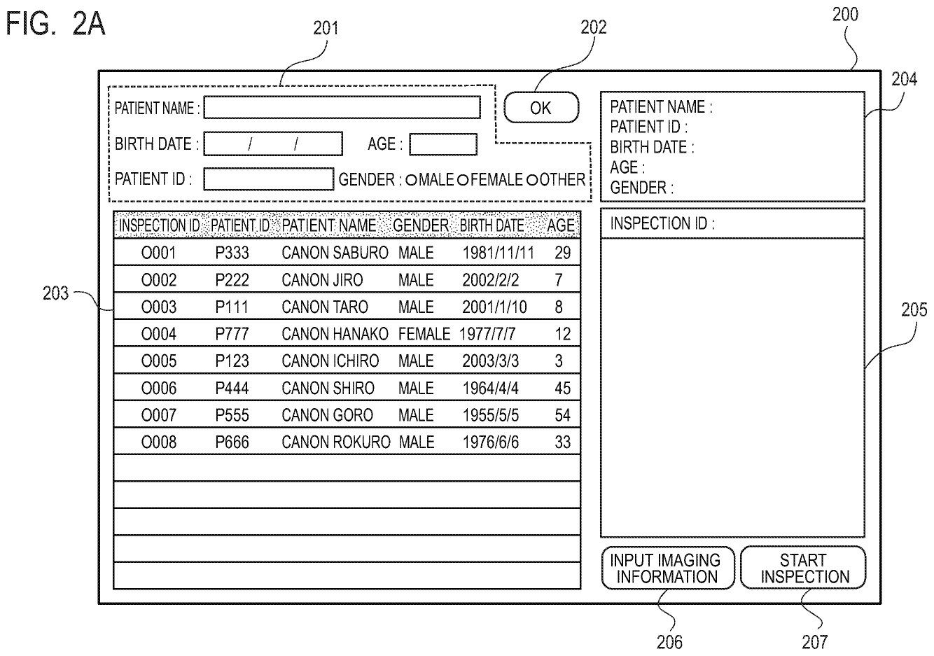

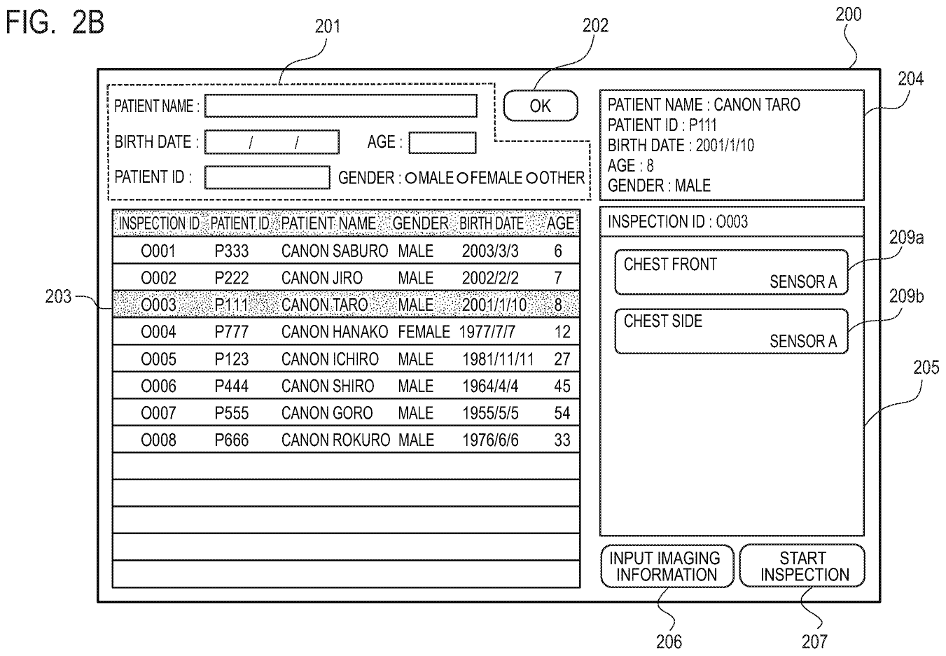

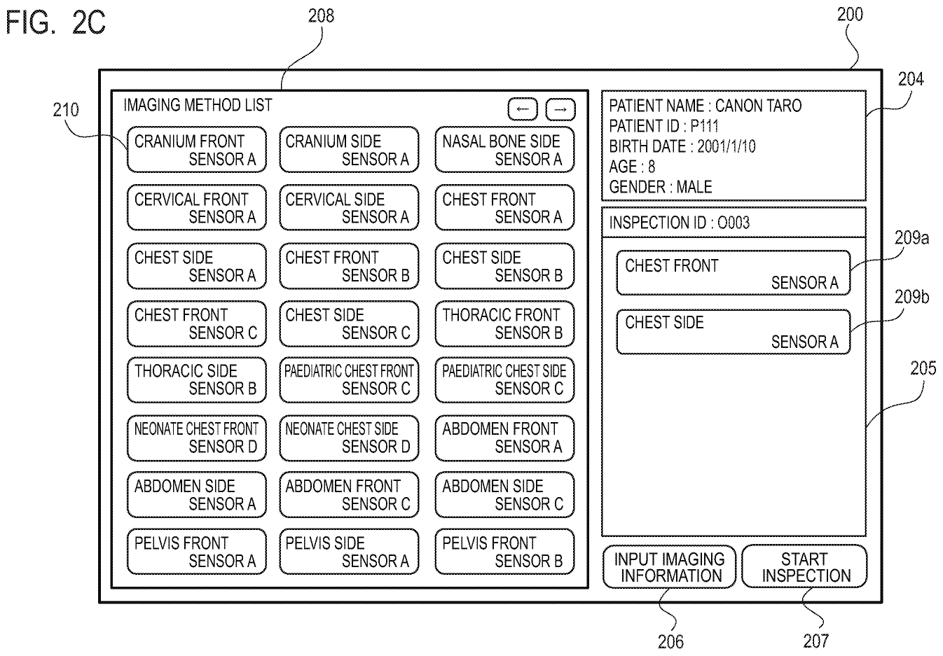

The CPU 111 of the imaging control apparatus 110 controls the display portion 115 to display a new inspection input screen 200. FIG. 2A is a diagram illustrating a display example of the new inspection input screen 200. As illustrated in FIG. 2A, the new inspection input screen 200 includes a patient information input area 201, a patient information determination button (OK button) 202, and a requested inspection list 203. The new inspection input screen 200 also includes a patient information display area 204, an imaging information display area 205, an imaging information input button 206, and an inspection start button 207.

In the requested inspection list 203, inspections received from the RIS 172 are arranged to be displayed as a list. When the operator selects any of the inspections from the requested inspection list 203, as illustrated in FIG. 2B, the patient information, e.g., patient ID, patient name, and birth date, on the selected patient is displayed in the patient information display area 204. In addition, an inspection ID is displayed in the imaging information display area 205, and the imaging information corresponding to the inspection ID is displayed in an area immediately below the inspection ID. The imaging information is received from the RIS 172 as described above. In the case of the example of FIG. 2B, imaging method buttons 209 (chest front button 209a and chest side button 209b) corresponding to the imaging information are arranged.

When the imaging information input button 206 is selected, as illustrated in FIG. 2C, an imaging information input area 208 is displayed to enable an imaging method to be added. In the example of FIG. 2C, a plurality of imaging method selection buttons 210 are displayed in the imaging information input area 208. The operator can add an imaging method by selecting one of the imaging method selection buttons 210. In this example, the added imaging method is displayed in the imaging information display area 205 in alignment with the chest front button 209a and the chest side button 209b. Each of the imaging methods is associated with an imaging method ID.

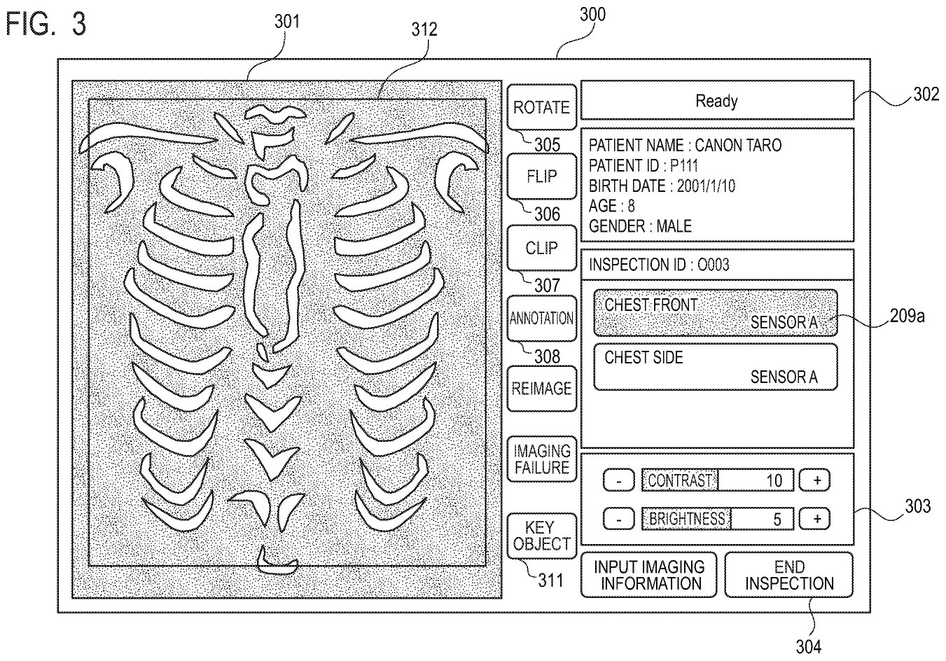

After confirming the patient information and the imaging information, the operator selects the inspection start button 207. The inspection to be performed is then determined. When the inspection start button 207 is selected, the imaging control apparatus 110 displays an imaging screen 300 illustrated in FIG. 3 on the display portion 115. The imaging screen 300 is a screen displayed at the time of imaging. The imaging screen 300 includes the same display areas as those of the new inspection input screen 200 described with reference to FIG. 2A to FIG. 2C. Newly-added display areas include an obtained image display area 301, a message area 302, an image processing setting area 303, and an inspection end button 304.

When the imaging screen 300 is displayed, the imaging method button 209 (chest front button 209a) arranged in the uppermost part in the imaging information display area 205 is in a selected state by default. In response to this selection, the CPU 111 of the imaging control apparatus 110 controls the radiation generation unit 120 based on imaging conditions, e.g., tube voltage, tube current, and irradiation time period, set for each imaging method button (imaging method). The CPU 111 then controls the radiation detector 140 based on the imaging conditions to prepare for imaging. When the preparation is completed, the imaging control apparatus 110 shifts to an imaging ready state. At this time, the CPU 111 displays, in the message area 302, a “Ready” message indicating that the imaging control apparatus 110 is in an imaging ready state.

The operator then views the imaging method to perform setting of imaging and positioning of the patient. After a series of imaging preparations are completed, the operator refers to the message area 302 to confirm that the imaging control apparatus 110 is in an imaging ready state, and then selects a radiation irradiation switch (not illustrated). In response, the imaging control apparatus 110 causes the radiation generation unit 120 to irradiate the object, i.e., patient's specific body part, with radiations, and causes the radiation detector 140 to detect the radiation that has passed through the object. The radiation image is thus obtained.

After the imaging is completed, the CPU 111 of the imaging control apparatus 110 acquires the obtained image from the radiation detector 140, and then performs image processes on the acquired image based on a predetermined image processing condition defined for each imaging method in advance. After the image processes are finished, the CPU 111 displays, in the obtained image display area 301, the obtained image subjected to the image processes. Changing of a contrast and other factors of the obtained image is accomplished by operating buttons for a contrast, a brightness, and other factors, which are provided in the image processing setting area 303.

Changing a clipping area of an output image is accomplished by operating, for example, a clipping button 307 and a clipping frame 312 to specify a desired clipping area. When a character string being diagnosis information is to be added, the operator operates, for example, an annotation button 308 to superimpose a graphic object, a character string, and other such annotations onto the image. When the orientation of the image is not suitable for diagnosis, the operator uses, for example, a rotation button 305 and a flip button 306 to perform geometric conversion. Using the above-described approaches, the operator can perform additional image editing on the obtained image displayed in the obtained image display area 301.

The operator repeats the above-described procedure to perform imaging operations corresponding to all the imaging methods included in the imaging information display area 205. When all imaging operations are finished, the operator selects the inspection end button 304. This ends a series of inspections, and the CPU 111 of the imaging control apparatus 110 re-displays the new inspection input screen 200. At this time, the imaging control apparatus 110 outputs a diagnostic image not considered to be an imaging failure to, for example, the PACS 173, the printer 174, or the ROM 112 after adding the inspection information, the imaging conditions, and other such information to the diagnostic image as supplementary information. The CPU 111 stores the obtained image and the patient information in the ROM 112 or another component in association with each other. In the above description, the obtained image is an example of the medical image.

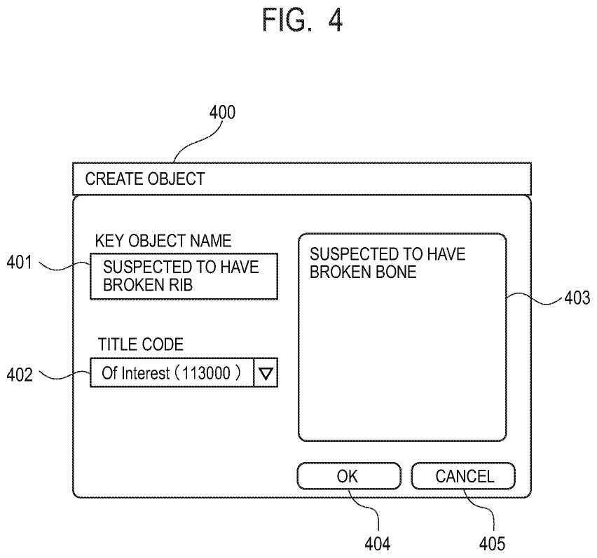

Next, a processing procedure in which the imaging control apparatus 110 creates a key object having a DICOM format is described. After confirming the obtained image on the imaging screen 300, the operator creates a key object as the requirement arises. When a key object button 311 is selected, a key object creation screen 400 illustrated in FIG. 4 is displayed. The key object creation screen 400 includes a key object name setting area 401, a title code setting area 402, and a remark input area 403.

The operator operates the key object name setting area 401 to set the name of a key object. The operator operates the title code setting area 402 to set a title code. In the title code setting area 402, title codes defined by the DICOM standard are registered in advance. An original title code that does not exist in the DICOM standard can also be added. The operator enters description or other such information about the key object in the remark input area 403. When the description or other such information about the key object is not required and only classification suffices, the operator is not required to specify the information.

When the operator selects an OK button 404 after setting information required for the key object, the CPU 111 of the imaging control apparatus 110 generates the key object set in the key object creation screen 400. When a cancellation button 405 is selected, the CPU 111 discards the key object being set. When the OK button 404 or the cancellation button 405 is selected, the CPU 111 closes the key object creation screen 400 to display the imaging screen 300. The key object is an example of a medical image associated object.

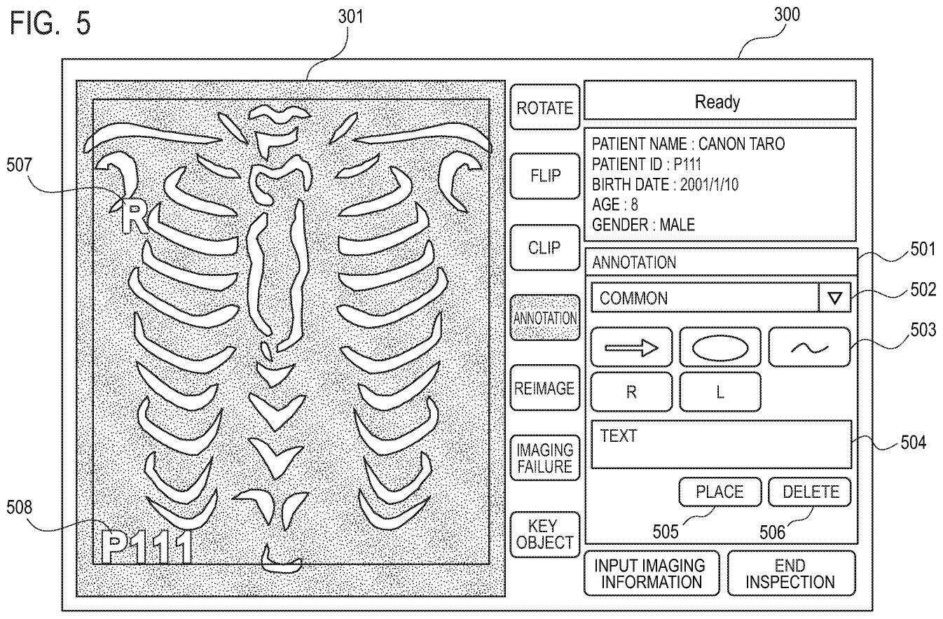

Next, a processing procedure in which the imaging control apparatus 110 places an annotation along the inspection flow is described. After the key object is created, the operator places an annotation as the requirement arises. When the annotation button 308 is selected, an annotation setting area 501 illustrated in FIG. 5 is displayed on the imaging screen 300. The annotation setting area 501 includes a group selection area 502, a graphic object placement button 503, a text input area 504, a text placement button 505, and an annotation deletion button 506.

The operator operates the group selection area 502 to set a group having the annotation. The operator can select, in the group selection area 502, a common group and a key object name of the key object created on the key object creation screen 400. When the setting in the group selection area 502 is completed, only the annotations within the group set in the group selection area 502 are displayed in the obtained image display area 301. However, the annotations in the common group can be displayed at all times. In this case, the common group is a group having the annotations within all the other groups. That is, when the common group is set for a given annotation, all the groups have the given annotation. In this manner, a plurality of annotations are grouped.

The operator selects the graphic object placement button 503 of a type desired to be placed. In response, the CPU 111 of the imaging control apparatus 110 displays an annotation as illustrated as a graphic annotation 507 so as to be superimposed onto the image. When text is placed as an annotation, the operator inputs a character string to the text input area 504 and selects the text placement button 505. In response, the CPU 111 of the imaging control apparatus 110 displays an annotation as illustrated as a text annotation 508 so as to be superimposed onto the image. The CPU 111 also stores the placed annotations in the storage portion of the ROM 112 or another component in association with the group selected in the group selection area 502.

The operator can select a placed annotation to move the annotation to any position in the obtained image display area 301. The operator can also select an undesired annotation and select the annotation deletion button 506 to delete the selected annotation from within the obtained image display area 301. The operator can change the group having the annotation by selecting an annotation for which the group is desired to be changed and changing the group set in the group selection area 502.

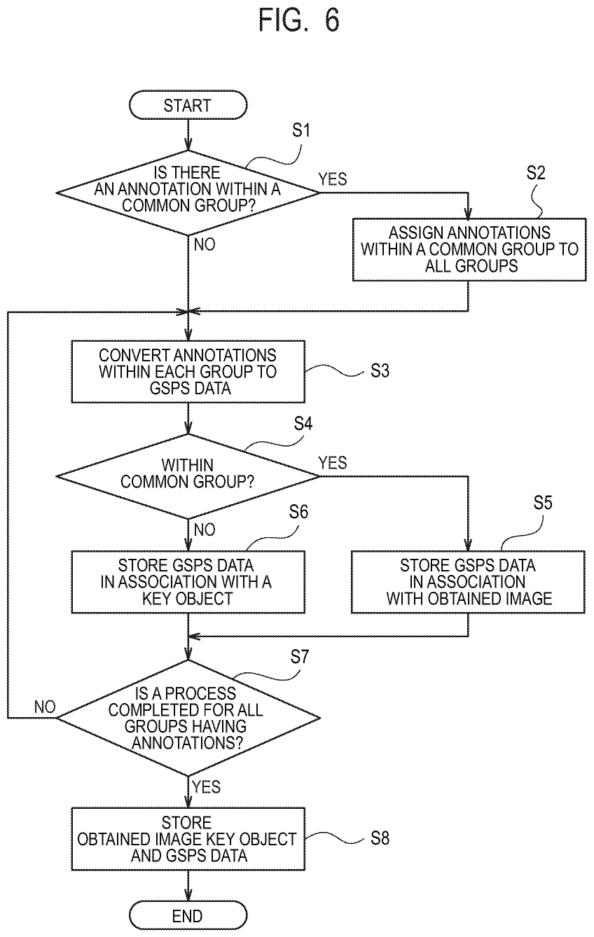

Next, a Grayscale Softcopy Presentation State (GSPS) creation process to be performed by the imaging control apparatus 110 is described. This process is an example of a medical image process. FIG. 6 is a flow chart of a GSPS creation process. First, in step S1, the CPU 111 of the imaging control apparatus 110 determines whether there is an annotation within the common group among annotations added to a medical image. When there is an annotation within the common group (YES in Step S1), the process proceeds to step S2. When there is no annotation within the common group (NO in step S1), the process proceeds to step S3.

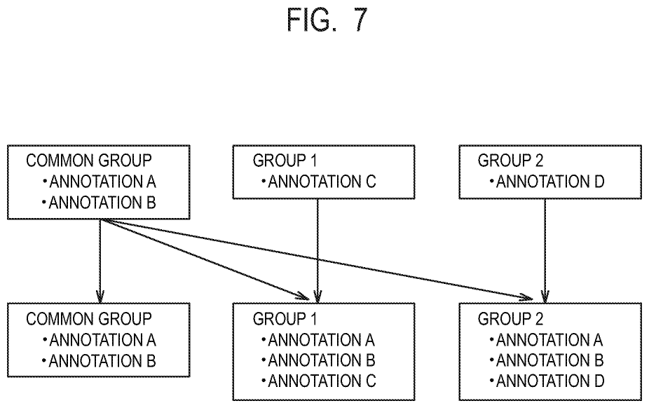

In step S2, the CPU 111 assigns the annotations within the common group to all the other groups. The process of step S2 will be described in detail with reference to FIG. 7. FIG. 7 is a diagram for illustrating an example of an annotation group. With reference to FIG. 7, the GSPS creation process is described specifically. It is assumed that, for example, four annotations A to D are added to the obtained image. It is also assumed that, as illustrated in the upper section of FIG. 7, the common group has the annotation A and the annotation B. It is assumed that a group 1 has the annotation C, and a group 2 has the annotation D.

The annotation A and the annotation B within the common group are assigned to all the groups other than the common group, namely, to the group 1 and the group 2, in the process of step S2. Thus, as illustrated in the lower section of FIG. 7, the group 1 has the annotation A, the annotation B, and the annotation C. In addition, the group 2 has the annotation A, the annotation B, and the annotation D.

Referring back to FIG. 6, after the process of step S2, in step S3, the CPU 111 converts annotations within each group into GSPS data in units of groups. Next, in step S4, the CPU 111 determines whether the annotation being the source of the GSPS data is within the common group. When the annotation is within the common group (YES in step S4), the process proceeds to step S5. When the annotation is not within the common group (NO in step S4), the process proceeds to step S6.

In step S5, the CPU 111 stores the GSPS data in association with the obtained image. Thus, the annotation within the common group is displayed simultaneously with the display of the obtained image. In step S6, the CPU 111 stores the GSPS data in association with the key object. Thus, when the associated object is selected based on the user operation, the CPU 111 can display the annotation within the group. After the processes of step S5 or step S6, the process proceeds to step S7.

In step S7, the CPU 111 determines whether the conversion into GSPS data has been completed for all the groups to be processed. When there is an unprocessed group (NO in step S7), the process returns to step S3. When the process has been completed for all the groups (YES in step S7), the process proceeds to step S8. In step S8, the CPU 111 stores the obtained image, the key object, and the GSPS data in, for example, the PACS 173, the ROM 112, or another component. The GSPS creation process then ends.

When the CPU 111 receives the designation of the annotation group based on the user operation in the reception process, the CPU 111 can display the annotations within the designated group on the display portion 115. This process is an example of a display control process.

As discussed in the above-described embodiment, in the imaging control apparatus 110 it is possible to automatically assign one annotation to all the groups without manually performing the process of assigning one annotation to each of the plurality of groups. Thus, the annotations within the common group are reflected in all pieces of GSPS data. That is, annotations can be efficiently grouped based on purposes.

In another exemplary embodiment, the common group, which is assumed to be applied to all the groups used for the medical image, can be applied to a plurality of groups, and groups to which the common group is applied are not limited to all the groups.

In still another exemplary embodiment, the CPU 111 can automatically assign a predetermined annotation to the common group. For example, the CPU 111 automatically assigns, to the common group, annotations that can cause inconsistency when individually placed in respective groups, for example, laterality markers for discriminating between left and right.

Embodiment(s) of the present disclosure can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present disclosure has been described with reference to exemplary embodiments, it is to be understood that these exemplary embodiments are not limiting. The scope of the following claims is to be accorded the broadest interpretation so as to encompass all such modifications and equivalent structures and functions.

This application claims the benefit of Japanese Patent Application No. 2019-143768, filed Aug. 5, 2019, which is hereby incorporated by reference herein in its entirety.

What is claimed is:

1. A medical image diagnosis system comprising:
a generation unit configured to generate a medical image associated object relating to a medical image;
a first associating unit configured to associate the medical image associated object with the medical image;
a grouping unit configured to assign a plurality of annotations to be added to the medical image to two or more groups; and
a second associating unit configured to associate each of the two or more groups with the medical image associated object,
wherein the grouping unit is configured to assign one of the plurality of annotations to a plurality of groups among the two or more groups.

2. The medical image diagnosis system according to claim 1, further comprising a reception unit configured to receive designation of a predetermined group,
wherein the grouping unit is configured to assign, when the predetermined group is designated, each of the plurality of annotations within the predetermined group to the two or more groups except the predetermined group.

3. The medical image diagnosis system according to claim 2, wherein the grouping unit is configured to assign, when the predetermined group is designated, each of the plurality of annotations within the predetermined group to all of the two or more groups.

4. The medical image diagnosis system according to claim 1, wherein the grouping unit is configured to assign a predetermined annotation to the predetermined group.

5. The medical image diagnosis system according to claim 1, further comprising a display control unit configured to display, when one of the two or more groups is designated based on a user operation, each of the plurality of annotations within the designated group on a display unit.

6. The medical image diagnosis system according to claim 1, wherein the medical image associated object is associated with the medical image as a key object of Digital Imaging and Communications in Medicine.

7. The medical image diagnosis system according to claim 1, further comprising a conversion unit configured to convert each of the plurality of annotations into a Grayscale Softcopy Presentation State of Digital Imaging and Communications in Medicine.

8. A medical image processing method executed by a medical image diagnosis system, the medical image processing method comprising:
a generating step of generating a medical image associated object relating to a medical image;
a first associating step of associating the medical image associated object with the medical image;
a grouping step of assigning a plurality of annotations to be added to the medical image to two or more groups; and
a second associating step of associating each of the two or more groups with the medical image associated object,
wherein the grouping step comprises assigning one of the plurality of annotations to a plurality of groups among the two or more groups.

9. A non-transitory computer-readable storage medium storing a program for causing a computer to execute a medical image processing method, the medical image processing method comprising:
a generating step of generating a medical image associated object relating to a medical image;
a first associating step of associating the medical image associated object with the medical image;
a grouping step of assigning a plurality of annotations to be added to the medical image to two or more groups; and
a second associating step of associating each of the two or more groups with the medical image associated object,
wherein the grouping step comprises assigning one of the plurality of annotations to a plurality of groups among the two or more groups.
